(19) United States
(12) Patent Application Publication
(10) Pub. No.: US 2005/0084552 A1
(43) Pub. Date: Apr. 21, 2005

(54) Pharmaceutical composition of chinese herbs for treating snuffle, headache and the use thereof

(75) Inventor: Wei Xiao, Xinpu District, Lianyungang Jiangsu (CN)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Jianqsu Kanion Pharmaceutical Co., LiLianyungang, Jiangsu (CN)

(21) Appl. No.: 10/497,287

(22) PCT Filed: Nov. 29, 2001
(86) PCT No.: PCT/CN01/01591

(51) Int. Cl. A61K 35/78

(52) U.S. Cl. 424/773

(57) ABSTRACT

This invention has published a composition of medicine for awaking brain, relieving pain and growing in intelligence, which is made up with dahurian angelica root 10-30 pare-by-weight, rhizome of Sichuan lovage 0.7-1.5 pare-by-weight, herba asari 0.7-1.5 pare-by-weight, borneolum syntheticum 0.7-1.5 pare-by-weight. The invented medicine has the feature of high absorbing speed, good effect, non-side-effect and easy administration. It can both treat disease and maintain health. Also, it is snuffed by nose, which makes it easily to administration.

### FIELD OF THE INVENTION

This invention is about a composition of medicine extracted from herbs for treating nasal obstruction and headache and its process of preparation.

### BACKGROUND OF THE INVENTION

The nasal obstruction and headache caused by wind and cold are common disease with high incidence in the winter and spring. The pathogenesis of the headache is that: the wind evil with cold evil infracts the head, so that the lucid yang can't circuit normally, the Qi and blood stagnate and block-in the channels of the head. If the wind and cold infract the lung, the lung can't exhale and inhale normally and the Qi of the lung can't move smoothly, so that the nasal obstruction occurs. The dahurian angelica root is pungent in flavor and warm in property. It is the herb belonging to the stomach meridian, large intestine meridian and the lung meridian. The pungent herb can dissipate the stasis and drive the Qi, so that dahurian angelica root has the capability to dissipate the wind and cold blocked in the head. The rhizome of Sichuan lovage is also pungent in flavor and warm in property, but it belongs to the liver meridian, the gallbladder meridian and the pericardium meridian. It has the function of promoting the circulation of Qi and blood and alleviating pain. The modern pharmacy researches indicate that: the dahurian angelica root includes the ingredients of imperatorin, isoimperatorin, angelicin, dahurian angelica ether. These ingredients are the main effective ingredients to assuaging pain, allaying excitement, relieveing inflammation and antisepsis. The rhizome of Sichuan lovage has the ingredients of ligustrazine and many phenols such like alkaloid and ferulic acid. These ingredients are the main effective mass to active the blood and relieve pain.

### SUMMARY OF THE INVENTION

This invention aims to supply a composition of medicine extracted from herbs for treating nasal obstruction and headache and its process of preparation, which has the feature of high absorbing speed, good effect, non-side-effect and easy administration. Another aim of the invention is to supply this herbs combination's new pharmaceutical applications.

The aim of the invention can be realized by the technical proposal as following:

Dahurian angelica root 400-2000 part-by-weight

Rhizome of Sichuan lovage 100-500 part-by-weight

The dahurian angelica root and rhizome of Sichuan lovage are smashed to pieces at the diameter of 0.3-1 cm. 4-8 times weight of water is added into the herbs to distill the naphtha. The residual water extract is concentrated to thick paste at the density no less than 1.30 at 60° C. Then the thick paste is extracted by the refluxing alcohol for 1 hour. The alcohol extract is filtrated to get a filtrated liquor. 10-16 times weight of alcohol is added into the distilled residue to extract for 1 hour refluently. The new extract is filtrated to get another filtrated liquor. The 2 shares of filtrated liquor are mixed together and concentrated in the low pressure to thick paste at the density no less than 1.30 at 60° C. The thick paste is dehydrated at 70° C. in vacuum. The dry medicine is smashed into powder and added into the naphtha. The medicine combination extracted on the process recorded can be made into any drug form needed in the clinic with the different supplementary materials. Every 0.54 g prepared material is equal to 1.41 g crude drugs.

The administration of the capsule produced on the invented method is 3 capsules one time and 3 times a day. The time of slaking after eating is 4-6 min and the ammidin concentration is 0.517-0.569%. Comparing with the existing medicine, the invented capsule has the feature of bringing into play in a shorter time, better curative effect and lower dose for oral administration and easier to administrate.

The invented herbs combination has been proved to be effective by the experiments and the preparation method has been optimized on the index of crude drugs content in the invented medicine. The crude drugs content in the extract prepared by the invented method can reach 17 g/ml. The invented medicine was proved by the State Administration of Traditional Chinese Medicine, Tianjin medicine research institute to have the functions of antisepsis, relieving the inflammation, relieving the pain, improving the microcirculation and lowering the blood viscosity.

### EXPERIMENT EXAMPLE 1

Experiment Medicine

The invented herbs extract is the brown yellow liquid, at the content 8 g/ml (the content of crude drugs, the same below) with the lot number 930821 and produced by Lianyungang Kangyuan Pharmacy Ltd Company. The medicine liquid is preserved in the refrigerator and diluted into the right concentration with the distilled water before the animal experiment.

The Experiment Animal

Kunming white mouse: weighing 18-22 g; the total number was 140; the sex was not limited.

Japanese white rabbit: weighing 2.2-3.2 kg; the total number was 42; the sex was not limited.

All the animals were supplied by Tianjin medicine research institute animal department; all the animals were raised in the observation room at the temperature of 26±2° C.; the animal certificate of approval number was “Jin 001”.

The Experiment Bacteria
staphylococcus aureus (6538)
type II hemolytic streptococcus (32210)
bacillus pyocyaneus (27853)

All the bacteria were supplied by Tianjin disease prevention center.

The Experiment Method and Result

(I) The Effect to Expel the Evil—Bacteriostasis Experiment In Vitro

The petri-dish 2-time-dilution method was used in the experiment. According to the routine manipulation of the microbion experiment, the staphylococcus aureus, type II hemolytic streptococcus and bacillus pyocyaneus were proliferated in the fitting culture fluid. All the bacteria were counted and diluted into solution of cfu/ml. The extract of Duliang was sucking out accurately and mixed into the nutrient agar uniformly at 50° C. to get nutrient petri dishes with different concentration of medicine. The bacteria solutions were sucked and smeared on the surface of the petri dishes. The bacteria in petri dishes were cultured in the constant-temperature-box at 37° C. for 48 hours, and then the growth of the bacteria were observed. The medicine concentration of the petri dish without living bacteria was recorded as the lowest antibacterial concentration and the petri dish without medicine was set as the negative control sample. The result showed that the invented medicine had bacteriostastic activity to all the 3 bacteria in the experiment (see table 1). The lowest antibacterial concentrations were 0.29/ml, 0.04 g/ml and 0.5 g/ml separately.

TABLE 1
The result of the bacteriostasis experiment in vitro

| | Bacteria | Medicine | |
|---|---|---|---|
| | amount | concentration | Bacteriostast-ic |
| Bacteria | (cfu/ml) | (g/ml) | activity |
| staphylococcus | 2.0 × 10⁵ | 0.8 | + |
| aureus (6538) | | 0.4 | + |
| | | 0.2 | + |
| | | 0.1 | − |
| | | 0.16 | + |
| type II hemolytic | 1.6 × 10⁵ | 0.08 | + |
| streptococcus | | 0.04 | + |
| (32210) | | 0.02 | − |
| bacillus pyocyaneus | 7.0 × 10⁵ | 1.0 | + |
| (27853) | | 0.5 | + |
| | | 0.25 | − |

(II) The Anti-Inflammatory Action—The Effect on the Mouse's Auricle Inflammation caused by Croton Oil

The male mice weighing 19-22 g were randomly divided into 4 groups and one of the groups was control group. Each group had 10 mice. The 3 treatment groups were feed the invented medicine on the dose of 8 g/kg, 4 g/kg and 2 g/kg once a day for 3 days by the stomach tube, the control group was feed the same volume of the distilled water by the same way. 30 minutes after the last administration, all the mice were smeared with 3% croton oil 0.02 ml on the left ears to induce the inflammation. 2 hours later, all the mice were killed and extirpated the both ears. Each mouse's ears were scissored into rondure on the diameter of 0.7 cm. The scissored ears were weighed accurately and the weight difference was recorded as the swelling degree. Comparing the swelling degree of the treatment group with the control group, the result indicated that feeding the invented medicine on 8 g/kg and 4 g/kg could inhibit the inflammation significantly, which was proved by t test. The group fed with the invented medicine on 8 g/kg had a very significant difference to the control group (P<0.001), while the group on the dose of 2 g/kg had no significant difference to the control group. The result showed that the invented medicine had the ability to relieve the inflammation.

TABLE 2
The effect of the invented medicine on the ear swelling of
the mouse (X ± SD)

| Group | Dose (g/kg) | Animal number | Swelling degree (mg) |
|---|---|---|---|
| Control group | | 10 | 8.70 ± 3.47 |
| Treatment group | 8 | 10 | 2.30 ± 1.95*** |
| (high dose) | | | |
| Treatment group | 4 | 10 | 4.30 ± 3.56* |
| (middle dose) | | | |
| Treatment group | 2 | 10 | 8.00 ± 3.97 |
| (low dose) | | | |

Note:
Comparing with the control group:
*P < 0.05
**P < 0.01
***P < 0.001 (the same below)

(III) Abirritation—The Effect on the Mouse's Whipping-Tail-Time Related to the Pain caused by Heating 20 male mice and 20 female mice weighing 19-22 g were divided into 4 groups at random. Each group was half male and half female. One group was set as control group. The other 3 groups were fed with the extract of the invented medicine on the dose of 8 g/kg, 4 g/kg and 2 g/kg. In the experiment, the mice in the treatment group were fed the medicine one time and the mice in the control group were fed with the distilled water on the same volume. Before the administration and 30 minutes, 60 minutes, 120 minutes after the administration, all the mice were test the whipping-tail-time (the whipping-tail-time was the time quantum which began from the mouse's tail putting into the hot water at the temperature of 55° C.±0.5° C. and ended at the mouse's tail whipping from the water.). The time difference before and after the administration were recorded and compared with the control group by the t test. The result indicated that the invented medicine could delay the whipping-tail-time significantly comparing to the control group at all the 3 doses (P<0.01, P<0.001). The experiment result showed that the invented medicine had significant antalgic function at all the 3 doses 8 g/kg, 4 g/kg and 2 g/kg (see table 3).

TABLE 3
The effect of the invented medicine to the mouse's whipping-tail-time(X ± SD)

| | | | | The time difference after | | |
|---|---|---|---|---|---|---|
| | | | | the administration (s) | | |
| | Dose | Animal | The whipping-tail-time before | 30 min after the | 60 min after the | 120 min after the |
| Group | (g/kg) | number | the administration(s) | administration | administration | administration |
| Control group | 0 | 10 | 1.146 ± 0.339 | −0.163 ± 0.373 | 0.020 ± 0.413 | −0.057 ± 0.353 |
| Treatment group | 8.0 | 10 | 1.110 ± 0.171 | 0.870 ± 0.626*** | 0.866 ± 0.539** | 0.953 ± 0.553*** |
| (high dose) | | | | | | |
| Treatment group | 4.0 | 10 | 1.114 ± 0.219 | 0.551 ± 0.636** | 0.506 ± 0.732 | 0.551 ± 0.408** |
| (middle dose) | | | | | | |
| Treatment group | 2.0 | 10 | 1.055 ± 0.311 | 0.420 ± 0.333** | 0.656 ± 0.623* | 0.579 ± 0.432** |
| (low dose) | | | | | | |

(IV) The Effect of the Invented Medicine on the Rabbits' Microcirculation and Blood Viscosity

Healthy rabbits were randomly divided into 3 groups. The sex was not limited. Each group had 8-10 rabbits. One group was the control group and the other 2 groups were treatment groups which were fed with the invented medicine on the doses of 4 g/kg and 2 g/kg by stomach tube once a day for total 3 days. 40 minutes after the last administration, all the rabbits were anesthetized with pentobarbitale sodium. The rabbits' bulbar conjunctiva microcirculation was observed with the stereo dissecting microscope at the magnification ratio 80. The number of the capillary meeting and the blood speed were recorded (fast→0, middle→1, slow→2, stop→3). The blood flow pattern was also recorded (straight line→0, broken line→1, flocculent state→2, stasis state→3). The 10% high molecular dextran was injected on the dose of 6 ml/kg to the rabbits' ear vessel to build the model with the microcirculatory disturbance and high blood viscosity. 20 minutes after the injection, the rabbits' bulbar conjunctiva microcirculation was observed for the second time. The number of the capillary meeting, the blood speed and the blood flow pattern were recorded again. 30 minutes after the injection, the blood was collected from the heart and preserved with heparin. The blood samples were measured with BME-1 blood viscometer (made in Shanghai) to get the data of the whole blood viscosity and blood plasma viscosity.

The result indicated that the invented medicine significantly improve the microcirculation disturbance caused by the high molecular dextran on the index of blood flow velocity, blood flow pattern and capillary meeting number (P<0.01). The results were showed in table 4 and table 5.

TABLE 4
The effect of the invented medicine on the microcirculation of the rabbit's
Bulbar conjunctiva(X ± SD)

| | | | Before the model building | | | After the model building | | |
|---|---|---|---|---|---|---|---|---|
| | Dose | Animal | capillary meeting | blood flow | blood flow | capillary meeting | blood flow | blood flow |
| Group | g/kg | number | number | velocity | pattern | number | velocity | pattern |
| Model control | 0 | 10 | 6.43 ± 1.34 | 0 | 0 | 3.83 ± 0.86 | 1.89 ± 0.35 | 1.89 ± 0.35 |
| group | | | | | | | | |
| Treatment group | 4 | 8 | 7.13 ± 1.13 | 0.06 ± 0.18 | 0.06 ± 0.08 | 6.75 ± 1.28*** | 0.69± 0.26*** | 0.69 ± 0.26*** |
| (high dose) | | | | | | | | |
| Treatment group | 2 | 8 | 6.80 ± 1.14 | 0.10 ± 0.21 | 0.10 ± 0.21 | 6.33 ± 1.41*** | 1.10 ± 0.57** | 1.10 ± 0.57** |
| (high dose) | | | | | | | | |

Note:
comparing with the control group,
*p < 0.05
**p < 0.01
***p < 0.01 (the same below).

TABLE 5
The effect of the invented medicine extract on the rabbit blood viscosity (X ± SD)

| | Dose | Animal | Whole blood viscosity | | | Blood plasma |
|---|---|---|---|---|---|---|
| Group | g/kg | number | Low tangent 20 S⁻¹ | Middle tangent 20 S⁻¹ | High tangent 80 S⁻¹ | viscosity |
| Control group | 0 | 6 | 8.41 ± 3.58** | 6.42 ± 1.14** | 4.50 ± 0.95** | 1.51 ± 0.24*** |
| Model control | 0 | 6 | 15.62 ± 3.90 | 8.91 ± 1.15 | 6.53 ± 1.03 | 2.89 ± 0.22 |
| group | | | | | | |
| Treatment | 4 | 6 | 7.29 ± 1.23*** | 6.05 ± 0.83*** | 5.25 ± 0.74* | 2.26 ± 0.29 |
| group (high | | | | | | |
| dose) | | | | | | |
| Treatment | 2 | 6 | 9.97 ± 3.04* | 6.99 ± 1.19* | 5.93 ± 1.07 | 2.45 ± 0.50 |
| group (low | | | | | | |
| dose) | | | | | | |

Conclusion

The experimental result proved that: the extract of the invented medicine could inhibit the growth of the 3 kind of bacteria:staphylococcus aureus, type II hemolytic streptococcus and bacillus pyocyaneus. The lowest bacteriostatic concentrations were 0.2 g/ml, 0.04 g/ml and 0.58/ml respectively. On the doses of 2 g/kg, 4 g/kg and 8 g/kg, the invented medicine could relieve the swelling degree of the mouse's ear caused by the croton oil and prolong the mouse's whipping-tail-time related to the pain caused by hot water. It proved that the invented medicine had the effect of anti-inflammation and abirritation. On the doses of 4 g/kg and 2 g/kg, the invented medicine could improve the microcirculatory disturbancy caused by the high molecular dextran and lower the blood viscosity.

In summary, the experiments proved that the invented medicine had the effects of bacteriostasis, anti-inflammation, abirritation and improving microcirculation. These experimental researches supplied the scientific proof to the medicine's clinical application. The invented medicine had been proved to be effective in treating the nasal obstruction and headache by the clinical trial.

### EXPERIMENT EXAMPLE 2

In a 100-patient clinical trial executed by Jiangsu TCM hospital, Nanjing TCM hospital and the People's hospital of North Jiangsu, the invented soft capsule's curative ratio and effective rate were 77% and 96% respectively.

(I) The Diagnostic Criteria of TCM

The headache is paroxysmal; usually the location is in the forehead, the temple and the occiput. The symptoms include nasal obstruction, aversion to cold and wind. The onset is quick and there are inducements such like fatigue and exposure to cold usually.

(II) The Methods and Contents about Observation

The observe about the curative effects included the symptoms' change such like headache, nasal obstruction, rhinorrhea, aversion to cold and wind, thirst or not, fur of tongue and pulse et al. The observe on the security included the indexes such like: physical examination, routine blood test, routine urine test, routine diachormea test, ECG, liver function test and kidney function test et al.

(III) Therapy

The patients in the treatment group took the invented soft capsule orally on the dose of 3 capsules one time and three times a day. 3-day-time was a period of treatment. The patients who were receiving the treating of the invented medicine were forbidden to use any other medicine with the similar effect.

(IV) The Curative Effect Criteria

1. The Criteria to Evaluating the Symptoms:

Headache:

| | |
|---|---|
| The degree is so heavy that the patient can't | 6 |
| continue the normal activity. | |
| The degree is middle; the patient can endure | 4 |
| and continue the normal activity. | |
| The degree is light and no activity is affected | 2 |
| by the headache. | |
| No symptom of headache | 0 |

2. The Criteria to Evaluate the Curative Effect

Based on the criteria to evaluating varies of indexes, the score before and after the treating were calculated. The ratio ( score before the treating/score after the treating) was used to evaluate the curative effect.

1) Clinical recovery: the ratio is 0-0.2.

2) Significantly effective: the ratio is 0.2-0.5.

3) Effective: the ratio is 0.6-0.8.

4) Ineffective: the ratio is bigger than 0.8.

(V) The Result:

In the 100-patient clinical trial, the invented soft capsule's curative ratio and effective rate were 77% and 96% respectively (table 6).

TABLE 6
The result of the 100-patient clinical trial

| | | | | | | Ratio of | |
|---|---|---|---|---|---|---|---|
| | | | | | | recovery | |
| | | | | | | and | |
| | | | Significantly | | | significantly | Ratio of |
| Group | Number | Recovery | effective | Effective | Ineffective | effective | effective |
| Treatment | 100 | 26 | 51 | 19 | 4 | 77.0% | 96.0% |
| group | | | | | | | |

### PRACTICE EXAMPLE 1

1130 g dahurian angelica root and 282 g rhizome of Sichuan lovage are crushed into pieces on the diameter of 0.5-1 cm and added with 6 times weight of water to distill the naphtha. The water extract is concentrated to thick paste with the relative density no less than 1.30 at 60° C. 14 times of alcohol is added to the thick paste and extracted refluxingly for 1 hour. The extract is filtrated to get the filtrated liquor. 14 times of alcohol is added to the solid residual of the herbs and extracted refluxingly for 2 times by heating. The residual-alcohol mixture is filtrated to collect the filtrated liquor. All the filtrated liquor are mixed and concentrated in low pressure to get thick paste on the density no less than 1.30 (60° C.). The thick paste is dehydrated in the vacuum (temperature<70° C.) and crushed to powder. The distilled naphtha and refined vegetable oil are added into the powder, then the mixture is milled by the colloid mill. The milled medicine powder is enclosed into the soft capsule to get the finished produce. Each soft capsule filled with drug is 0.54 g weight and equals to the crude drugs 1.41 g.

### PRACTICE EXAMPLE 2

1500 g dahurian angelica root and 375 g rhizome of Sichuan lovage are crushed into pieces on the diameter of 0.3 cm and added with 8 times weight of water to distill the naphtha. The water extract is concentrated to thick paste with the relative density no less than 1.342 at 60° C. 16 times of alcohol is added to the thick paste and extracted refluxingly for 1 hour. The extract is filtrated to get the filtrated liquor. 14 times of alcohol is added to the solid residual of the herbs and extracted refluxingly for 2 times by heating. The residual-alcohol mixture is filtrated to collect the filtrated liquor. All the filtrated liquor are mixed and concentrated in low pressure to get thick paste on the density no less than 1.30 (60° C.). The thick paste is dehydrated in the vacuum (temperature<70° C.) and crushed to powder. The distilled naphtha and refined vegetable oil are added into the powder, then the mixture is milled by the colloid mill. The milled medicine powder is enclosed into the soft capsule to get the finished produce. Each soft capsule filled with drug is 0.54 g weight and equals to the crude drugs 1.41 g.

1. A composition of medicine for treating nasal obstruction and headache made from the original herbs based on weight proportion as following:
dahurian angelica root 400-2000;
Rhizome of Sichuan lovage 100-500.

2. The composition of medicine according to claim 1 wherein dahurian angelica root is 1130, wherein Rhizome of Sichuan lovage is 282.

3. The composition of medicine according to claim 1 wherein dahurian angelica root is 1500, wherein Rhizome of Sichuan lovage is 375.

4. The composition of medicine according to claim 1, which can be made into any clinically accepted form.

5. A method for preparing the composition of medicine according to claim 1 comprising:
smashing the dahurian angelica root and Rhizome of Sichuan lovage to pieces at the diameter of 0.3-1 cm, adding into water 4-8 times weight of the herbs to distill the naphtha; concentrating the residual water extract to thick paste at the density no less than 1.30 at 60° C.; extracting the thick paste by the refluxing alcohol for 1 hour, filtrating the extract to get a filtrated liquor; adding 10-16 times of alcohol to the thick paste and extracting refluxingly for 1 hour, filtrating to get the filtrated liquor; adding 10-15 times of alcohol to the solid residual of the herbs and extracting refluxingly for 2 times by heating, filtrating and collecting the filtrated liquor; mixing the 2 shares of filtrated liquor and concentrating it in a reduced pressure to a thick paste at the density no less than 1.30 at 60° C.; dehydrating the thick paste below 70° C. in vacuum; smashing the dry medicine into powder and adding into the naphtha for the further process; the material extracted on the process recorded being made into capsule form with the different supplementary materials; every 0.54 g prepared material being equal to 1.41 g crude drugs.

6. The method for preparing the composition of medicine according to claim 5 comprising:
crushing dahurian angelica root and Rhizome of Sichuan lovage into pieces on the diameter of 0.5-1 cm and adding 6 times of water to distill the naphtha, concentrating the water extract to thick paste with the relative density no less than 1.30 at 60° C., adding 14 times of alcohol to the thick paste and extracting refluxingly for 1 hour, filtrating to get the filtrated liquor; adding 14 times of alcohol to the solid residual of the herbs and extracting refluxingly for 2 times by heating, filtrating and collecting the filtrated liquor; mixing all the filtrated liquor and concentrating it in the low pressure to a thick paste on the density no less than 1.30 at 60° C., dehydrating the thick paste in the vacuum below 70° C. and crushing it to the powder, adding the distilled naphtha and refined vegetable oil to the powder and milling the mixture to be uniform by the colloid mill, putting the milled medicine powder into the soft capsule to get the finished product.

7. The method for preparing the composition of medicine according to claim 5 comprising:
crushing 1500 g dahurian angelica root and 375 g Rhizome of Sichuan lovage into pieces on the diameter of 0.3 cm and adding 8 times of water to distill the naphtha, concentrating the water extract to a thick paste with the relative density no less than 1.342 at 60° C., adding 16 times of alcohol to the thick paste and extracting reflusingly for 1 hour, filtrating to get the filtrated liquor; adding 14 times of alcohol to the solid residual of the herbs and extracting reflusingly for 2 times by heating, filtrating and collecting the filtrated liquor; mixing all the filtrated liquor and concentrating it in the low pressure to a thick paste on the density no less than 1.30 at 60° C., dehydrating the thick paste in the vacuum below 70° C. and crushing it to the powder, adding the distilled naphtha and refined vegetable oil to the powder and milling the mixture to be uniform by the colloid mill, putting the milled medicine powder into the soft capsule to get the finished product.

8. A method for relieving inflammation or pain, comprising administering the composition of medicine according to claim 1 to a patient in need thereof.

9. A method for improving the microcirculation and lowering the blood viscosity, comprising administering the composition of medicine according to claim 1 to a patient in need thereof.

10. A method for treating headache, comprising administering the composition of medicine according to claim 1 to a patient in need thereof.
